(19) United States
(12) Patent Application Publication
(10) Pub. No.: US 2011/0250132 A1
(43) Pub. Date: Oct. 13, 2011

(54) LIPOIC ACID METABOLITE: USEFUL FOR DRUG CARRIER, NANOPARTICLE CONJUGATE, IMAGING AND HYPERTHERMIA THERAPY

(75) Inventor: Ravikumar Kabyadi Seetharama, Redwood City, CA (US)

(73) Assignee: Ravikumar Kabyadi Seetharama, Redwood City, CA (US)

(21) Appl. No.: 12/826,465

(22) Filed: Jun. 29, 2010

Related U.S. Application Data
(60) Provisional application No. 61/341,975, filed on Apr. 8, 2010.
(60) Provisional application No. 61/341,977, filed on Apr. 8, 2010.
(60) Provisional application No. 61/343,069, filed on Apr. 23, 2010.

(51) Int. Cl. A61K 51/04; A61K 39/395; A61K 38/00; A61K 38/08; A61K 31/555; A61P 43/00; C07K 2/00; C07K 16/00; C07F 1/12; C07F 1/00; A61P 35/00; A61B 5/055; A61K 31/28

(52) U.S. Cl. 424/1.65; 424/9.32; 424/9.6; 424/133.1; 514/19.3; 514/21.7; 514/184; 514/495; 530/300; 530/391.7; 534/10; 544/225; 549/3; 556/113; 977/906; 977/773

(57) ABSTRACT

The present invention relates to simultaneous tracking or imaging followed by hyperthermia therapy for cancer and other diseases associated with altered metabolic enzymes. In particular, the invention relates to a novel class of therapeutic nanomaterial which selectively target and kill tumor cells.

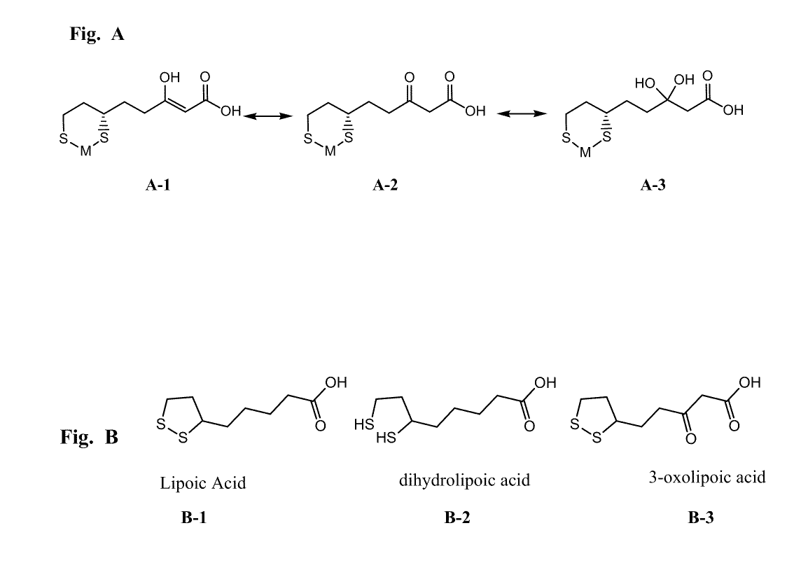

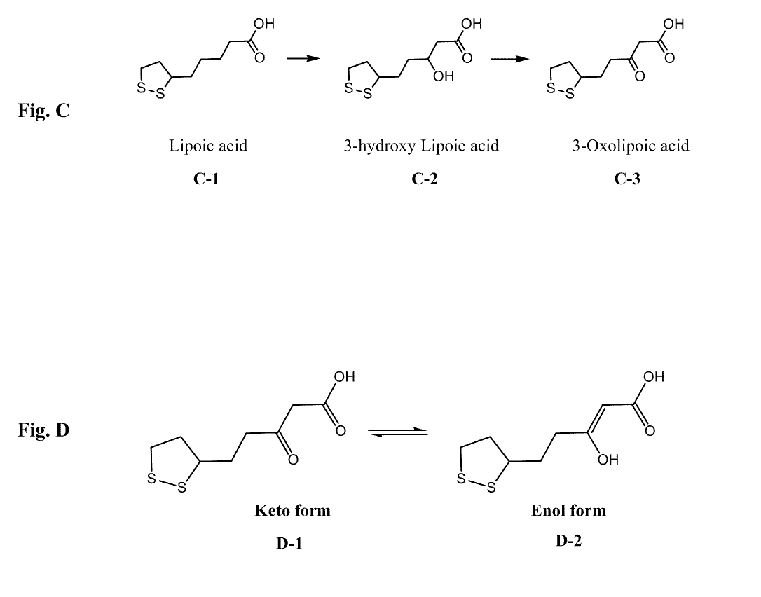

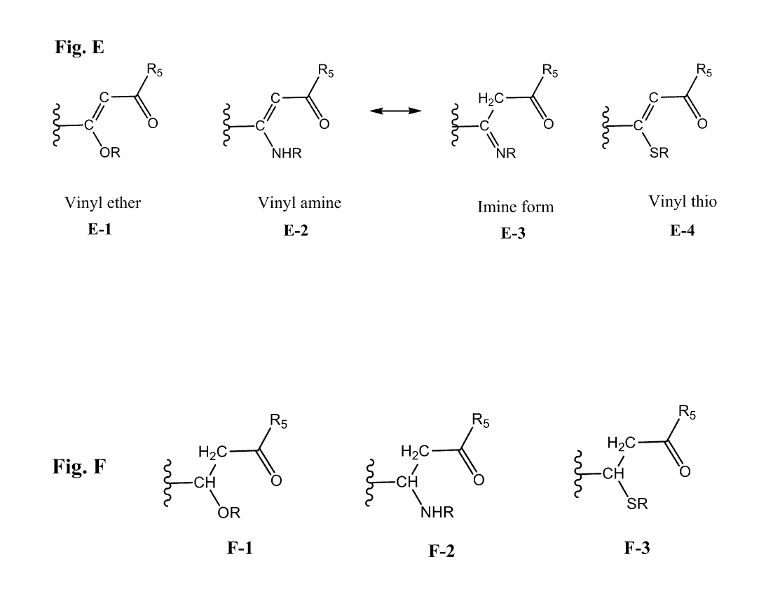

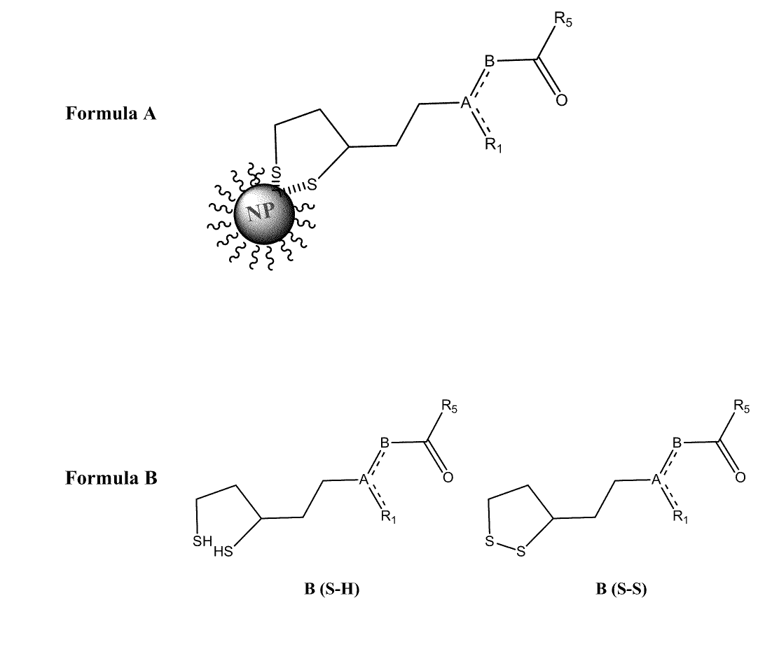

### CROSS-REFERENCE TO RELATED APPLICATIONS

This application claims priority to provisional patent applications U.S. Ser. No. 60/923,774 filed Apr. 17, 2007; 60/923,775 filed Apr. 17, 2007; 61/341,975 filed Apr. 8, 2010; 61/341,977 filed Apr. 8, 2010; and 61/343,069 filed Apr. 23, 2010 incorporated herein in their entirety by reference.

### FIELD OF THE INVENTION

The present invention relates to therapeutics and diagnostics for cancer and other diseases associated with altered metabolic enzymes. In particular, the invention relates to a novel class of therapeutic agents which selectively target and kill tumor cells, and certain other types of cells involved in disease processes.

The present invention relates to a receptor binding conjugate on nanosize Nanoparticle (NP) which consists of a small molecule, vitamin, carbohydrates, peptides, and Chemotherapeutic agent, wherein or not, the conjugate possesses dual binding ability.

The present invention discloses an iron doped gold nanoparticles, comprising: a Lipoic acid ligand, dihydrolipoic acid ligand and metabolite of lipoic acid ligand (3-hydroxydihydro lipoic acid and 3-oxodihydrolipoic acid) on the surface thereof, wherein the iron doped gold nanoparticles generates fluorescence by the interaction between the ligand and the nanoparticle and the particle diameter of the iron doped gold nanoparticle is between 3 to 70 nm and is used as bioprobes and/or applied in a fluorescent biological label, clinical image as contrast medium, clinical detection, clinical trace, and clinical treatment, etc.

The present invention discloses that 3-hydroxy group of lipoic acid or dihydrolipoic acid forms intermolecular hydrogen bonding which further assists the formation of self assembled monolayer.

The present invention discloses that 3-keto group of lipoic acid or dihydrolipoic acid exists in the form of dihydroxy group in aqueous media and also as keto-enol tautomers (Figure A) which facilitate for the formation of self assembled monolayer and decrease the formation of aggregation of nanoparticle.

The targeted delivery can be used, for example, for disease sensing, imaging, drug delivery, and hyperthermia therapy. The present invention also relates to a method and a kit to prepare, as well as a method to use such conjugates.

### BACKGROUND OF THE INVENTION

### Cancer

### General Description

Cancer can be defined as a disease that is characterized by a failure of the control mechanisms that are involved in cell division. It is very well known that the cancer cells have great appetite for food, particularly carbohydrate, vitamin and minerals. Carbohydrates play an important role in the human body, such as oligonucleotide synthesis, calcium (Ca⁺²), Lithium⁺¹, ion transport, etc. Complex organisms rely on communication between individual cells in order to maintain life functions. The development of new blood vessels, Angiogenesis, plays a central role in the pathogenesis of cancer. It is crucial for maintaining the supply of oxygen and nutrients to support tumor growth. Rapidly proliferating cells have a higher demand for DNA and consequently require large quantities of purine, pyrimidine, amino acids, and carbohydrates substrates.

For anti-tumor therapy, different strategies have been employed, e.g., radiotherapy, chemotherapy, or immunotherapy. Notably, these approaches do not only address the tumor cells themselves, but also the tumor stroma cells, e.g., endothelial cells, fibroblasts, and macrophages. This is of advantage since these cells actively contribute to the proliferative and invasive behavior of the tumor cells via secretion of growth factors, angiogenic factors, cytokines, and proteolytic enzymes. In addition, tumor stroma cells take part in immune evasion mechanisms of cancer. Thus, approaches targeting the tumor.

Stroma attracts increasing attention as anti-cancer therapy. Several molecules including growth factors (e.g., VEGF, CTGF), growth factor receptors (CD105, VEGFRs), adhesion molecules (αvβ3 integrin), and enzymes (CAIX, FAPa, MMPs, PSMA, uPA) are all expressed in tumor cell. Consequently, these molecules can be targeted by inhibitors as well as by active and passive immunotherapy to treat cancer. (Medicine in development of Cancer by Billy Tauzin, PhRMA, 2009, 1-104).

Many human cancer cell lines have been found to have highly over-expressed levels of the protein which binds folic acid. (Weitman, S. D., Lark, R. H., Coney, L. R., Fort, D. W., Frasca, V. Zurawski, Jr., V. R., and Kamen, B. A. Cancer Research 52, 3396-3401 (1992); Ross, J. F., Chaudhuri, P. K., and Ratnam, M. Cancer 73, 2432-2443 (1993); Prasad, P. D., Ramamoorthy, S., Moe, A. J., smith, C. H., Leibach, F. H., and Ganaphthy, V. Biochim. Biophys. Acta 1223, 71-75 (1994); Li, P. Y, Vecchio, S. D., Fonti, R., carriero, M. V., Potena, M. I., Botti, G., Miotti, S., Lastoria, S., Menard, S., Colnaghi, M. I., and Salvatore, M. J. Nuclear Med. 37, 665-672 (1996); Antony, A. C. Annu Rev. Nutr. 16, 501-521 (1996); Bueno, R., Appasani, K., Mercer, H., Lester, S., and Sugarbaker, D. J. Thoracic Cardio. Sur. 121, 225-233 (2001)). These facts have attracted considerable attention (Reddy, J. A. and Low, P. S. Critical Reviews in Therapeutic Drug Carrier Systems 15, 587-627 (1998); Drummond, D. C., Hong, K., Park, J. W., Benz, C. C., and Kirpotin, D. B. Vitamins and Hormones 60, 285-332 (2001); Sudimack, J. B. A. and Lee, R. J. Advanced Drug Delivery Reviews 41, 147-162 (2000)) and have been exploited for developing cancer selective drug delivery system (DDS) (Lee, R. J. and Low, P. S. J. Biol. Chem. 269, 3198-3204 (1994); Lee, R. J. and Low, P. S. Biochim. Biophys. Acta 1233, 134-144 (1995); Rui, Y., Wang, S., Low, P. S., and Thompson, D. H. J. Am. Chem. Soc. 120, 11213-11218 (1998); Goren, D., Horowits, A. T., Tzemach, D., Tarshish, M., Zalipsky, S., and Gabizon, A. Clinical Cancer Research 6, 1949-1957 (2000)).

A number of researchers have compared the number of folate-receptors on malignant tissue versus normal tissue. (Mantovani, L. T.; Miotti, S.; Menard. S.; Canevari, S.; Raspagliesi, F.; Bottini, C.; Bottero, F.; Colnaghi, M. I.; Eur. J. Cancer 30A, 363-369 (1994); Ross, J. F.; Chaudhuri, P. K.; Ratnam, M. Cancer, 73, 2432-2443 (1994). It shows that malignant ovarian, endometrial, and brain tissues all have significantly higher numbers of folate receptors than normal tissues. Folate receptors have also been detected in breast, lung, colon, kidney, and head/neck cancers.

### Paramagnetic Iron and Gold Nanoparticles—General Description

Gold Nanoparticles for health-promoting and disease-preventing uses have been around for a long time. Suspension of colloidal gold can still be found in the dietary supplements. Gold nanoparticle size can be tuned reliably and routinely from 1 nm to 200 nm. Once the particle is at the target tissue, release of the payload of heat is induced by plasmonic heating. To afford the degree of precision required for tumor targeting, and to achieve the correct excitation wavelength, it is envisaged that the source of light would need to be a laser. Gold nanoparticles are sufficiently small that, in principle, they might be capable of delivering a payload of heat, directly into the cytoplasm or nucleus of the target cell which causes irreversible thermal cellular destruction.

Nanoparticles of gold have characteristic absorption bands in the electronic spectra because of Plasmon resonance. (Link, S.; El-Sayed, M. A. J. Phys. Chem. B 2001, 105, 1; Link, S; El-Sayed, M. A. Int. Rev. Phys. Chem. 2001, 19, 409; Mulvaney, P. Langmuir, 1996, 788, 12). Gold nanoparticles can absorb or scatter light at a desired wavelength across visible and NIR wavelength. The nanoparticle converts absorbed light to heat with an efficacy and stability that far exceeds that of conventional fluorescent dyes. (Pissuwan, D.; Valenzuela, S. M.; Cortie, M. B.; Trends in Biotechnology, 24(2), 62-67 (2006).

Magnetic nanoparticles offer some attractive possibilities in biomedicine. First, they have controllable sizes ranging from a few nanometers up to tens of nanometers, which places them at dimensions that are smaller than or comparable to those of a cell (10-100 um), a virus (20-450 nm), a protein (5-50 nm) or a gene (2 nm wide and 10-100 nm long). This means that they can ‘get close’ to a biological entity of interest. Second, these nanoparticles are magnetic. Magnetic nanoparticles have found utility in numerous biological applications, including sensing (Perez, M. J.; Josephson, L.; O'Loughlin, T.; Hogemann, D.; Weissleder, R. Nat. Biotechnology, 2002, 20(8), 816), imaging (Huh, Y. M; Jun, Y. W.; Song, H. T.; Kim, S,; Choi, J. S.; Lee, J. H.; Yoon, S.; Kim, K. S.; Shin, J. S.; Suh, J. S.; Cheon, J. J. Am. Chem. Soc., 2005, 127, 12387), hyperthermia for tumor therapy (Shinkai, M.; Yanase, M.; Suzuki, M.; Honda, H.; Wakabayashi, T.; Yoshida, J.; Kobayashi, T. J. Magn. Magn. Mater. 1999, 194, 176-184).

Hamad-Schifferli et al published magnetic field heating study of iron doped gold nanoparticles encapsulated with bis(p-sulphonatophenyl)phenylphosphine dihydrate (Wijaya, A.; Brown, K. A.; Alper, J. D. Hamad-Schifferli, K J. Magn. Magn Mater. 2007, 309, 15-19). Willner et al used maghemite-Au core-shell nanoparticle/polyaniline composites for the study of magnetoswitchable charge transport and bioelectrocatalysis (Riskin, M.; Basnar, B.; Willner, I. Adv. Mater. 2007, 10, 2691). Recent publication from University of texas southwestern medical center showed the preparation and evaluation of a radioisotope-incorporated iron oxide core/Au shell nanoplatform for dual modality imaging (Zhou, Y-F et al. J. Biomedical Nanotechnology, 2008, 4(4), 474).

### Lipoic Acid and Its Metabolite—General Description

Alpha-lipoic acid was first isolated by Reed and coworkers as an acetate replacing factor. It is slightly soluble in water, and soluble in organic solvents. Alpha-lipoic acid is a chiral molecule and is known by a variety of names including thioctic acid; 1,2-diethylene-3-pentanoic acid; 1,2-diethylene-3-valeric acid; and 6,8-thiooctic acid. Alpha-lipoic acid was tentatively classified as a vitamin after its isolation, but it was later found to be synthesized by animals and humans. The complete enzyme pathway that is responsible for the de novo synthesis has not yet been definitively elucidated.

More recently, a great deal of attention has been given to possible antioxidant functions for alpha-lipoic acid, and its reduced form, dihydrolipoic acid (DHLA). Lipoate, or its reduced form, DHLA, which reacts with reactive oxygen species, such as superoxide radicals, and singlet oxygen. It also protects membranes by interacting with Vitamin C and glutathione, which may in turn recycle vitamin E.

One of the strongest naturally occurring antioxidants is lipoic acid (LA). α-Lipoic acid is also known as thioctic acid. LA in its reduced form (dihydrolipoate, DHLA) possesses two —SH groups which provide a very low oxidation potential to the molecule (−0.29 v). Thus, LA and the DHLA redox together are excellent antioxidants capable of interacting with most forms of reactive oxygen species. LA is one of the most important molecules in redox signaling due to maintenance of oxidizing conditions by stabilizing disulfides in the extra cellular surface while the intracellular environment is maintained in the reduced state with the help of free sulfhydryl groups.

There is evidence that the two optical isomers of alpha-lipoic acid have different biological activities. R-alpha-lipoic acid occurs naturally in plants and animals and is the only form that functions as a cofactor for mitochondrial enzymes. Chemical synthesis of alpha-lipoic acid results in a 50/50 or racemic mixture of S-alpha-lipoic acid and R-alpha-lipoic acid. Within the mitochondria, R-alpha-lipoic acid is reduced to DHLA, the more potent antioxidant, 28 times faster than S-alpha-lipoic acid. However, in the cytosol S-alpha-lipoic acid is reduced to DHLA twice as fast as R-alpha-lipoic acid. One study in humans found R-alpha-lipoic acid to be more bioavailable than S-alpha-lipoic acid when taken orally. R-alpha lipoic acid was more effective than S-alpha-lipoic acid in enhancing insulin-stimulated glucose transport and metabolism in insulin-resistant rat skeletal muscle, and R-alpha-lipoic acid was more effective than racemic alpha-lipoic acid and S-alpha-lipoic acid in preventing cataracts in rats.

Lipoic acid readily crosses the blood-brain barrier and accumulates in all neuronal cell types (Packer, L.; Tritschler, H. J.; Wessel, K. Free Radic. Biol. Med. 22, 359-378 (1997). There, cytosolic and mitochondrial dehydrogenases rapidly reduce it to dihydrolipoic acid (DHLA).

More recently, Goralska and co-workers reported beneficial effects of lipoic acid treatment in preventing iron accumulation in lens epithelial cells (Goralska, M; Dackor, R.; Holley, B.; McGahan, M. C. Exp. Eye Res. 76, 241-248 (2003). Thus, it is plausible that lipoic acid may also be beneficial in normalizing the adverse effects or iron accumulation in the aging brain.

Rao et al. studied the novel effects of metal ion Chelation on the properties of lipoic acid-capped silver and gold nanoparticles. (Berchmans, S.; Thomas, P. J.; Rao, C. N. R. J. Phys. Chem. B 2002, 106, 4647-4651).

Porta et al reported that Au(0) nanoparticles, stabilized by 5-amino valeric acid, selectively penetrate into K562 cancer cells in a short time. These experiments were carried out in order to verify the specific recognition of gold sol by abnormal cells. The observed selectivity towards gold nanoparticles by K562 makes the metallic system attractive for cancer therapy (Krpetic, Z.; Porta, F. Scarf, G. Gold Bulletin, 2006, 39(2), 66-68).

### Hyperthermia—General Description

Hyperthermia is one of the promising approaches in cancer therapy (Kobayashi, T.; J Hyperthermic Oncol. 1993, 9, 245). Various methods are employed in hyperthermia, such as whole body hyperthermia (Cavaliere, R; Ciocatto, E. C.; Giovanella, B. C. Cancer, 1967, 20, 1351), radiofrequency hyperthermia (Ikeda, N.; Hayashida, O.; Kameda, H. Int. J. Hyperthermia, 1994, 10, 553) or inductive hyperthermia (Lin, J. C.; Wang, Y. J. Int. J. Hyperthermia, 1987, 3, 37). However, the inevitable technical problem with hyperthermia is the difficulty of uniform heating of only the tumor region until the required temperature without damaging normal tissue.

Some researchers have proposed intracellular hyperthermia and developed submicron magnetic particles for hyperthermia (Mitsumori, M.; Hiraoka, M.; Shibata, T.; Int. J. Hyperthermia, 1994, 10, 785). These magnetic particles are easily incorporated into cells and generate heat under an alternating magnetic field. Especially, the colloidal magnetic iron oxide is metabolized and excreted from the body, so that they are more ideal materials than the others.

Dewhirst and Needham published liposomal drug delivery with hyperthermia. (Tashjian, J. A.; Dewhirst, M. W.; Needham, D.; Viglianti, B. L. Int. J. Hyperthermia, 2008, 24(1), 79-90; Chen, Q.; Krol, A.; Wright, A.; Needham, D.; Dewhirst, M. W.; Yuan, F. Int. J. Hyperthermia, 2008, 24 (6), 475-482.

For the avoidance of doubt, the compounds of lipoic acid and its metabolite has the formula as shown in Figure B.

It is believed that lipoic acid initially oxidized in vivo to 3-hydroxy lipoic acid which further oxidized to 3-oxo lipoic acid as shown in Figure C.

The present inventions identify the keto-enol tautomers of the metabolite of lipoic acid (Figure D) and use these tautomers for effective encapsulation of nanoparticle.

The present invention uses the ene-derivative of the lipoic meteabolite as shown in Figure E.

The present invention uses the reduced form of ene-derivative of the lipoic acid as shown in Figure F.

The compounds of the present invention are those compounds of Formula A.

The compound of the present invention disclose that the nanoparticle was encapsulated with the ligand of the Formula B.

### SUMMARY OF THE INVENTION

One aspect of the present invention is directed towards a multivalent product with iron doped gold nanoparticle coupled by the ligands.

Another aspect of the present invention is directed to a method of making the multivalent product. This method includes providing iron doped gold nanoparticle and the ligand.

A further aspect of the present invention is directed toward a method of imaging. The present invention may be useful in providing targeting and imaging capabilities in the treatment of tumors and other abnormalities of tissue. The construct is designed to consist of a self assembled monolayer of metal complexes on a metal core. The self assembled monolayer is achieved by the attachment of iron doped gold nanoparticle metal surface with thiol or disulfide groups. The formation of a self assembled mono layer of nanoparticle metal core provides uniformity in the chemical and physical properties of the construct that are important to its targeting and imaging characteristics.

The present invention provides a novel class of compounds and it may be used in the development of in vitro diagnostic reagent.

The present invention provides a novel class of compounds and it may be used in the development of in vivo diagnosis.

According to the present invention, the composition includes gold nanoparticles and the ligand of the Formula B.

According to the present invention, the composition includes iron doped gold nanoparticles encapsulated with the ligand of the Formula B.

The present invention provides a novel class of compounds and it may be used in a method of treating various pathologies in a subject. The class of compounds comprises iron doped gold nanoparticles encapsulated with the ligand of the Formula B and pharmaceutically acceptable salts thereof.

The carrier particle of the immunogenic composition can include a material selected from the group consisting of iron and gold. The carrier particle of the immunogenic composition can include a nanoparticle that has a diameter of from about 1 nm to about 1000 nm, a diameter of less than or equal to about 100 nm, or a diameter of greater than about 100 nm. The carrier particle of the immunogenic composition can be substantially biologically inert.

The immunogenic composition can have a ratio of antigenic conjugate molecules bound to the carrier particle in the range of from about 20:1 to about 1:20. For example, the immunogenic composition can have a ratio of antigenic molecules bound to the carrier particle of about 1:1. For example, the total number of antigenic conjugate molecules bound to the carrier particle can be in a range of from about 2 to about 100.

The current invention may be used in the main areas of biomedical applications including MRI contrast agents, for use as potential hyperthermia treatment of malignant cells, targeted drug delivery, and also as tools to manipulate cell membranes.

One preferred class of compounds comprise the structure of Formula A wherein: NP is gold nanoparticle; iron doped gold nanoparticle compound of the Formula A wherein S is bonded to one or more nanoparticle (NP) or direct bond between the two S atom bind to one or more nanoparticle (NP); and

A and B are carbon atoms directly connected to carbon-carbon single bond, carbon-carbon double bond.
R₁ is (═O); —OH; —OR₂; —OR₃R₄; OR₄; —NH, —NR₂, —NR₃R₄, —NR₄, ═NH; ═NR₂, ═NR₃R₄; ═NR₄; —NHOH, —NOR₂, —NOR₃R₄, —NHOR₄, ═N—OH; ═NOR₂; ═NOR₃R₄; ═NOR₄; NHNHR₂; NHNHR₃R₄; NHNHR₄; NR₂SO₂R₂; NR₂SO₂R₃R₄; NR₂SO₂R₄;
R₂ denotes COCH₃; —COCHCl₂; —COC₆H₅, -alkyl CₙH₂ₙ₊₁; -alkene CₘH₂ₘ; -alkyne CₘH₂ₘ₋₂; —(CH₂—CH₂O)ₙ CₘH₂ₘ₊₁; —(CH₂CH₂CH₂O)ₙ CₘH₂ₘ₊₁; —X(CH₂—CH₂O)ₙ CₘH₂ₘ₊₁Y; X(CH₂CH₂CH₂O)ₙ CₘH₂ₘ₊₁Y; n is 0-16; m is 0-16; X is CO, COO, CH₂O, CONHNH; Y is OH, NH₂, SH, COOH, SO₃H, H₂PO₄; R₃ denotes (CH₂)ₘ (XCH₂CH₂)ₙY; —(CH₂)ₘ (XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH;
R₄ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol, Bevacizumab;
R₅ is OH; OLi, ONa; OK, 0Mg, OMn, OCu, OCa, OAl, OFe, OAg, OAu, O-Niacinamide salt, O-Thiamine salt, ZR₆, ZR₇R₈, ZR₈, NR₉SO₂R₁₀ and Z is O, S, NH, NHNH. R₆ denotes -alkyl CₙH₂ₙ₊₁; -alkene CₘH₂ₘ; -alkyne CₘH₂ₘ₋₂; —(CH₂CH₂O)ₙ CₘH₂ₘY; —(CH₂CH₂CH₂O)ₙ CₘH₂ₘY; n is 0-16; m is 0-16; Y is H, OH, NH₂, SH, COOH;
R₇ denotes (CH₂)ₘ (XCH₂CH₂)ₙY; —(CH₂)ₘ (XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH;
R₈ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol, Bevacizumab;
R₉ denotes R₆, R₇R₈, R₈; and
R₁₀ denotes R₆, R₇R₈, R₈.

### BRIEF DESCRIPTION OF THE DRAWINGS

Fig. A is the present invention keto-enol tautomers.

Fig. B is the compound of lipoic acid and metabolites.

Fig. C shows metabolites of lipoic acid.

Fig. D shows the keto-enol tautomers of the metabolite of lipoic acid.

Fig. E is the ene-derivatives of the metabolites of lipoic acid.

Fig. F is the reduced form of the ene-derivatives.

Formula A shows the compounds of the present invention.

Formula B shows the encapsulating ligands of the present invention.

### DETAILED DESCRIPTION OF THE

### Preferred Embodiments

### Structural Characteristics of Nanomaterial Conjugate

The iron doped gold nanoparticle may have a shape which is spherical, rod shaped, or polyhedral.

One aspect of the present invention is iron doped gold nanoparticle core encapsulated with ligands.

The ligands may be derived from the Formula B.

The product may also include plurality of polyethylene glycol (PEG) molecules attached to the iron doped gold nanoparticle. PEG molecules helps to fill the free spaces left after the adsorption of ligand.

The product may also include plurality of pentane thiol molecules attached to the iron doped gold nanoparticle. Pentane thiol molecule helps to fill the free spaces left after the adsorption of ligand.

A further aspect of the present invention is directed toward a method of imaging. The method of imaging may include magnetic resonance imaging (MRI), fluorescence imaging, surfaced-enhanced Raman imaging, radiologic imaging or the targeted delivery of radioisotopes. The targeted delivery of radioisotopes may include targeted delivery of radioisotopes to tissues, such as tumors.

The method of imaging may include imaging carried out in conjunction with a real-time MRI or CT guided procedure.

The nanomaterial conjugate of the present invention embraces that compound of the formula A wherein S is bonded to one or more nanoparticles or direct bond between the two S atom bound to one or more nanoparticles.

The alkyl group preferably comprises CₙH₂ₙ₊₁ wherein n is 0-16. Such alkyl groups may be substituted with the moieties such as, for example, OH, NH₂, and Cl. Examples of alkyl groups include, but are not limited to, methyl, ethyl, butyl, and decanyl.

An alkene may preferably comprise CₙH₂ₙ wherein n=2-16. Examples of alkene groups include, but are not limited to, propylene and cyclopropene.

The alkyne may preferably comprise CₙH₂ₙ₋₂ wherein n is 2-16. Examples of alkyne groups include, but are not limited to, propyne and acetylene.

Alkyl, alkene and alkyne groups can have additions on any of their carbons. Examples of additions include, but are not limited to, hydroxyls and amines.

### DEFINITIONS

The term “lipoic acid' is intended to mean α-lipoic acid which is a chiral molecule also known as thioctic acid; 1,2-diethylene-3 pentanoic acid; 1,2-diethylene-3 valeric acid; and 6,8-thioctic acid. Unless specified, the term covers the racemic mixture as well as any other (non-50-50) mixture of the enantiomers including substantially pure forms of either the R-(+) or the S-(−) enantiomer. Further, unless specified otherwise, the term covers pharmaceutically acceptable salts (e.g. Na and K salts) and amides, esters and metabolites of the acid.

The terms “treating”, “treatment”, and the like, are used herein to generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment”, as used herein, covers any treatment of a disease in a mammal, particularly a human, and includes: (a) preventing the disease from occurring in a subject which may be predisposed to the disease, but has not yet been diagnosed as having it; (b) inhibiting the disease, i.e. arresting it's development; or (c) relieving the disease, i.e. causing regression of the disease and/or it's symptoms or conditions. The invention is directed towards treating patients suffering from disease related to abnormal tissues. The present invention is involved in preventing, inhibiting, or relieving adverse effects attributed to cancer tissues.

The term “excipient material” is intended to mean any compound forming a part of the formulation which is intended to act merely as a carrier, i.e. not intended to have biological activity itself.

The term “chemical degradation” is intended to mean that the lipoic acid active ingredient is subjected to a chemical reaction which disrupts its biological activity.

The terms “synergistic”, “synergistic effect”, and the like are used interchangeably herein to describe improved treatment effects obtained by combining diagnosis followed by hyperthermia therapy of the invention with chemotherapeutic agent. Although a synergistic effect in some fields means an effect which is more than additive (e.g., 1+1=3), in the field of treating cancer and related diseases an additive (1+1=2) or less than additive (1+1=1.5) effect may be synergistic.

The term “quick release formulation” refers to a conventional oral dosage formulation. Such a formulation may be a tablet, capsule or the like designed to provide for substantially immediate release of the active ingredient and includes enteric coated oral formulation which provides some initial protection to the active ingredient and thereafter allow substantially immediate release of the entire active ingredient. A quick release formulation is not formulated in a manner so as to obtain a gradual, slow, or controlled release of the active ingredient.

### Methods for Using Nanomaterial

The nanomaterial of the present invention may be used in a method for preventing or inhibiting diseases involving altered or distinct cellular activity. Such diseases are characterized by sensitivity to the lipoate compositions of the present invention. One of the most important advantages of our nanomaterial is non-toxic or reduced toxicity of nanomaterial and simultaneous tracking or imaging the cells for the regional selective hyperthermia. Additional advantage is from the synergistic effect by the combination of hyperthermia with chemotherapeutic agent. Cells with appropriately altered are particularly targeted and killed, while surrounding healthy tissues remain unharmed by the nanomaterial.

In a preferred treatment method, the instant nanomaterial compositions are used for the preventing and treatment of cancers, such as primary or metastatic melanoma, thymoma, lymphoma, Hodgkin's lymphoma, leukemias, uterine cancer, cervical cancer, bladder cancer, kidney cancer, colon cancer, and adenocarcinomas such as breast cancer, prostate cancer, ovarian cancer, and pancreatic cancer. A wide variety of tumor types, including cervical carcinomas and breast cancers, are sensitive to this new class of compounds.

The preferred dosage of the nanomaterial, or pharmaceutical composition thereof, is selected based on other criteria, including the particular composition employed and the age, weight, and condition of the individual. Importantly, the quantity of nanomaterial derivative used should be sufficient to inhibit or kill tumor cells while leaving normal cells substantially unharmed.

By adapting the treatments described herein, the nanomaterial derivatives may also be used in methods for treating diseases other than cancer, where the disease-causing cells exhibit altered metabolic patterns. For example, eukaryotic pathogens of humans and other animals are generally much more difficult to treat than bacterial pathogens because eukaryotic cells are so much more similar to animal cells than are bacterial cells. Because of the multi action of the nanomaterial to treat cancer tissues in the present invention and antibacterial properties of gold nanoparticle, these nanomaterials will prove to be of therapeutic importance of a new class of antibacterial agent.

Another important application of this nanomaterial may be used as diagnostic agents in vitro and in vivo. As stated earlier, depending on the specific tumor cell or cell type in question, different nanomaterial conjugates may be more or less effective at inhibiting distinct tumor classes. Thus, for example, in cases where diagnosis or selection of an appropriate hyperthermia therapy and chemotherapeutic strategy may be difficult, testing of a culture of tumor cells in vitro with nanomaterial known to target specific tumor cell types, provides an alternative approach for identifying tumor types and effective treatments.

### Compositions of Nanomaterial for Therapeutic Use

In the methods of preventing or inhibiting cancer, the nanomaterial derivative, or a pharmaceutical composition comprising a nanomaterial derivative, may be administered via one of several routes including intravenous, intramuscular, subcutaneous, intradermally, intraperitoneal, intrathoracic, intrapleural, intrauterine, topical, or intratumor.

Those skilled in the art will recognize that the mode of administering the nanomaterial depends on the type of cancer, or symptom to be treated. For example, a preferred mode of administering the nanomaterial for treatment of leukemia would involve intravenous administration, whereas preferred methods for treating skin cancer would involve, for example, topical or intradermal administration.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the methods and materials are now described.

Before the present, formulations, methods and components used therein are disclosed and described, it is to be understood that this invention is not limited to particular compounds, excipients or formulations as such may of course, vary. It is also to be understood that the terminology used herein is for the purpose of describing particular embodiments only, and is not intended to be limiting, since the scope of the present invention will be limited only by the appended claims.

For therapeutic applications, a pharmaceutical composition comprising an effective amount of the nanomaterial described above, along with pharmaceutically acceptable carrier, is administered directly to a patient. The compositions may be in the form of tablets, capsules, powders, granules, suppositories, reconstitutable powders, or liquid preparations, such as oral or sterile parenteral solutions or suspensions. However, for consistency of administration, it is preferred that the nanomaterial is in the form of unit dose. For oral administration, tablets and capsules may contain conventional excipients, such as binding agents, tabletting lubricants, and pharmaceutically acceptable wetting agents, such as sodium lauryl sulphate.

For parenteral administration, fluid unit dosage forms are prepared utilizing the nanomaterial and a sterile vehicle, and, depending on the concentration used, can either be suspended or dissolved in the vehicle. In preparing solutions, the nanomaterial can be dissolved in water for injection and filter sterilized before filling into a suitable vial or ampoule and sealing. Also, adjuvant such as local anesthetic, a preservative, and buffering agents can be dissolved in the vehicle. To enhance stability, the composition can be frozen after filling into the vial and the water removed under vacuum. Parenteral suspensions are prepared in substantially the same manner, except that the nanomaterial is suspended in the sterile vehicle. A surfactant or wetting agent can be included in the composition to facilitate uniform distribution of the nanomaterial.

### Experimental Procedure

Synthesis of NHS Ester of Lipoic acid (LA-NHS): To a stirred solution of Lipoic acid (10.3 g, 50 mmol) in dry acetonitrile (50 mL) were added N,N′-disuccinimidyl carbonate (19.2 g, 75 mmol) and triethylamine (21 mL, 150 mmol). The resulting mixture was stirred at 25° C. for 2 hrs. The mixture was concentrated under reduced pressure and the residue was diluted with aqueous NaHCO₃ solution (100 mL) and extracted thoroughly with Ethyl acetate (3×75 mL). The combined extracts were washed with brine and dried over anhydrous MgSO₄ and filtered. Evaporation of the solvent provided the activated ester as a pale yellow solid (11.4 g, 77%).

Synthesis of Lipoic hydrazide (LA-NHNH₂): Hydrazine hydrate (5 mL) in MeOH (50 mL) was added to a solution of NHS lipoate (3.03 g, 10 mmol) in MeOH (50 mL), and the solution was stirred at room temperature for 4 hrs. After vacuum evaporation of most of the solvent, the residue was taken in CHCl₃. Washed with 5% NaHCO₃ solution, dried over anhydrous MgSO₄, filtered and evaporated the solvent to afford lipoic hydrazide (1.6 g, 73%).

Synthesis of Dihydro lipoic acid (DHLA): Lipoic acid (4.12 g, 20 mmol) was dissolved in 90 mL of 0.25 M NaHCO₃ solution and cooled to 0° C. in an ice bath. NaBH₄ (3.8 g, 100 mmol) was added slowly in such a way that the temperature maintained between 0 to 4° C. After the complete addition of NaBH₄, the reaction mixture was warmed to room temperature and stirred further for 2 hrs. The reaction mixture was acidified with 6N HCl to pH 1 and extracted with toluene (3×50 mL). The combined organic phase was dried over MgSO₄ and filtered. Evaporation of the solvent yielded dihydrolipoic acid (4 g, 95%).

Synthesis of DHLA-NHS: To a stirred solution of dihydrolipoic acid (2.1 g, 10 mmol) in dry acetonitrile (20 mL) were added N,N′-disuccinimidyl carbonate (2.8 g, 11 mmol) and triethylamine (1.7 mL, 12 mmol). The resulting mixture was stirred at 25° C. for 2 hrs. The mixture was concentrated under reduced pressure and the residue was diluted with aqueous NaHCO₃ solution (100 mL) and extracted thoroughly with Ethyl acetate (3×75 mL). The combined extracts were washed with brine and dried over anhydrous MgSO₄ and filtered. Evaporation of the solvent afforded NHS dihydrolipoate (2.5 g, 82%).

Synthesis of DHLA-hydrazide: Hydrazine hydrate (2.5 mL) in MeOH (25 mL) was added to a solution of dihydro NHS-lipoate (1.53 g, 5 mmol) in MeOH (20 mL), and the solution was stirred at room temperature for 4 hrs. After vacuum evaporation of most of the solvent, the residue was taken in CHCl₃. The chloroform solution was washed with 5% NaHCO₃ solution, dried over anhydrous MgSO₄, filtered and evaporated the solvent to afford dihydrolipoic hydrazide (0.96 g, 86%).

Synthesis of 3-oxolipoic-NHS ester: To a stirred solution of 3-oxolipoic acid (11 g, 50 mmol) in dry acetonitrile (50 mL) were added N,N′-disuccinimidyl carbonate (19.2 g, 75 mmol) and triethylamine (21 mL, 150 mmol). The resulting mixture was stirred at 25° C. for 2 hrs. The mixture was concentrated under reduced pressure and the residue was diluted with aqueous NaHCO₃ solution (100 mL) and extracted thoroughly with Ethyl acetate (3×75 mL). The combined extracts were washed with brine and dried over anhydrous MgSO₄ and filtered. Evaporation of the solvent provided the activated ester as a pale yellow solid (11.9 g, 75%).

Synthesis of 3-hydroxydihydrolipoic acid: A solution of 3-oxolipoic acid (2.20 g, 10 mmol) and MP-borohydride (8 g, 20 mmol) in MeOH (100 mL) was stirred at room temperature for 24 hrs. The solution was filtered and the resin was washed with dichloromethane (2×20 mL) and the filtrates and washings were combined and evaporated to dryness (2.1 g, 94%).

Synthesis of ADMPA-NHS: To a stirred solution of 4-Amino-4-deoxy-N°-methylpteroic acid (6.5 g, 20 mmol) in dry DMF (50 mL) were added N,N′-disuccinimidyl carbonate (7.7 g, 30 mmol) and triethylamine (5.6 mL, 40 mmol). The resulting mixture was stirred at 25° C. for 4 hrs. The mixture was diluted with aqueous NaHCO₃ solution (500 mL) and extracted thoroughly with Ethyl acetate (3×75 mL). The combined extracts were washed with brine and dried over anhydrous magnesium sulfate and filtered. Evaporation of the solvent provided the activated ester (4.9 g, 58%).

Synthesis of ADMPA-Hydrazide: To a stirred solution of hydrazine hydrate (10 mL) in DMF (25 mL) was added ADMPA-NHS (0.422 g, 1 mmol) and the solution was stirred at room temperature for 16 hrs. The reaction mixture was filtered to remove any insoluble residue and the filtrate was saturated with MeOH to precipitate the product. The product was isolated by filtration, washed with MeOH, ether and dried under vacuum to afford ADMPA-hydrazide (0.31 g, 90%).

Synthesis of DHLA-ADMPA conjugate: A solution of dihydrolipoic hydrazide (2.44 g, 11 mmol) and ADMPA-NHS ester (4.2 g, 10 mmol) were taken together in DMF (30 mL) and stirred until complete disappearance of ADMPA-NHS ester. After 5 hrs of stirring, the reaction mixture was diluted with ice cold water (200 mL) and filtered. The resultant residue was purified through silica gel column chromatography to afford the pure conjugate as a pale yellow solid (3.85 g, 73%).

Synthesis of DHLA-Methotrexate conjugate: Methotrexate (9 g, 20 mmol) and O-(7-Azabenzotriazol-1-yl)-N,N,N′,N′-tetramethyluronium hexafluoro phosphate, HATU (11.4 g, 30 mmol), N-methyl Morpholine (6 g, 60 mmol) were taken together in acetonitrile (100 mL) and DMF (10 mL) and into this solution dihydrolipoic hydrazide (5.6 g, 25 mmol) was added and stirred for 16 hrs. The reaction was quenched with water and extracted with ethyl acetate (3×50 mL) and the organic layer was isolated, dried over anhydrous magnesium sulfate, filtered, and evaporated to dryness. The crude product was purified through silica gel column chromatography to afford α-conjugate (0.31 g, 24%) and w-conjugate (0.78 g, 59%) in 1:2.5 ratio.

Synthesis of 3-oxolipoic acid-cRGDfK conjugate: 3-oxolipoic acid (1.1 g, 5 mmol) and O-(7-Azabenzotriazol-1-yl)-N,N,N′,N′-tetramethyluronium hexafluoro phosphate, HATU (2.28 g, 6 mmol) were taken together in acetonitrile (25 mL) and into this solution a mixture of cRGDfK.2TFA (4.15 g, 5 mmol) and N-methyl morpholine (1.01 g, 10 mmol) was added and stirred for 16 hrs. The reaction mixture was quenched with water and the reaction mixture was extracted with ethyl acetate (3×50 mL). The combined organic layer was dried over anhydrous magnesium sulfate, filtered and evaporated to dryness. The crude product obtained was purified through silica gel column chromatography and the pure product containing fractions were combined and evaporated to dryness to afford pale yellow solid (3.3 g, 82%).

Synthesis of 3-hydroxydihydrolipoic acid-cRGDfK conjugate: 3-oxolipoic acid-cRGDfK conjugate (1.61 g, 2 mmol) was dissolved in 10 mL of 0.25 M NaHCO₃ solution and cooled to 0° C. in an ice bath. NaBH₄ (0.38 g, 10 mmol) was added slowly in such a way that the temperature maintained between 0 to 4° C. After the complete addition of NaBH₄, the reaction mixture was warmed to room temperature and stirred further for 16 hrs. The reaction mixture was acidified with 6N HCl to pH 1, cooled and filtered. The residue was washed with 2-propanol and the resultant residue obtained was dried under vacuum afforded the desired product as a buff color solid (1.45 g, 86%).

General Procedure for the Direct Coupling of Dihydrolipoic Acid with Amine Reactive Ligand

Method 1: Dihydrolipoic acid (1 equivalent) and amine reactive ligand (1.2 equivalent) were taken together in tetrahydrofuran and into this solution N,N′-diisopropyl carbodimide (1.2 eq) and diisopropyl ethyl amine (1.2 eq) were added and the reaction mixture was stirred for 5 to 12 hrs. The reaction mixture was quenched with water and stirred for 3 to 5 hrs. Filtered the solution and the filtrate was extracted with ethyl acetate and the organic layer was isolated and washed with water, dried over anhydrous magnesium sulfate and filtered. The filtrate was evaporated to dryness and the crude product was purified through silica gel column chromatography to afford the pure product.

Method 2: Dihydrolipoic acid (1 equivalent) and HATU were taken together in acetonitrile and into this solution amine reactive ligand (1.2 equivalent) and N-methyl morpholine (2 equivalent) were added and stirred for 6 to 16 hrs. The reaction mixture was quenched with water and the resultant mixture was extracted with ethyl acetate. The organic layer was isolated, dried over magnesium sulfate, filtered and evaporated to dryness to afford the desired conjugate.

### General Procedure for the Synthesis of Functionalized Gold Nanoparticle of Different Size

Method 1: An aqueous golden yellow solution of HAuCl₄ 3H₂O (3.39 g, 10 mmol) in water (50 mL) was mixed with a solution of tetradecylammonium bromide (13.2 g, 20 mmol) in toluene (100 mL). The two-phase mixture was vigorously stirred until all the tetrachloroaurate was transferred into to the organic phase. After 30 minutes the organic phase showed dark red in color and the aqueous phase colorless. Lipoic acid conjugate or dihydrolipoic acid conjugate (12 mmol) in acetonitrile or DMF was then added, causing the organic phase to become orange. A freshly prepared sodium borohydride (3.78 g, 0.1 mol) in water (100 mL) was slowly added (5 mL installment) with vigorous stirring causes color changing to dark brown with evolution of gas. After complete addition, the reaction mixture was stirred for 3 hrs at 25 to 100° C. and the organic phase was isolated and evaporated to dryness. The resulting brown powder obtained was redissolved in toluene (50 mL) and 200 mL of 2-propanol was added to precipitate the colloid and the product was collected by filtration. The residue was washed multiple times with 2-propanol to fully remove unreacted thiol.

Method 2: An aqueous solution of HAuCl₄ 3H₂O (0.68 g, 2 mmol) in water (50 mL) was brought to boil. Lipoic acid conjugate or dihydrolipoic acid conjugate (1.2 mmol) in acetonitrile or DMF was added into the above solution. A freshly prepared solution of trisodium citrate (3 to 10 mmol) in water (50 mL) was rapidly added into the above solution and the mixture refluxed for 30 minutes. The colloidal solution was cooled, filtered through a microporous filter (Millipore 0.22 μm filter) and centrifuged. The nanomaterial was resuspended in water and 2-propanol and centrifuged. The process of centrifugation and resuspension was carried out multiple times to fully remove unreacted thiol.

General Procedure for the Synthesis of Iron Doped Gold Nanoparticle of Different Size

Method 1: An aqueous golden yellow solution of HAuCl₄ 3H₂O (0.34 g, 1 mmol) and FeCl₃ (0.16 g, 1 mmol) in water (100 mL) was mixed with a solution of tetradecylammonium bromide (2.64 g, 4 mmol) in toluene or chloroform (100 mL). The two-phase mixture was vigorously stirred for 60 minutes. Lipoic acid conjugate or dihydro lipoic acid conjugate (12 mmol) was then added, causing the organic phase to become orange. A freshly prepared sodium borohydride (0.38 g, 10 mmol) in water (25 mL) was slowly added (5 mL installment) with vigorous stirring causes color changing to dark brown with evolution of gas. After complete addition, the reaction mixture was stirred for 3 hrs at 25 to 100° C. and the organic phase was isolated and evaporated to dryness. The resulting brown powder obtained was redissolved in toluene or chloroform (100 mL) and 2-propanol was added to precipitate the colloid and the product was collected by centrifugation and filtration. The residue was washed multiple times with 2-propanol to fully remove unreacted conjugate.

Method 2: An aqueous solution of HAuCl₄ 3H₂O (0.34 g, 1 mmol) and FeCl₃ (0.16 g, 1 mmol) in water (100 mL) and brought to boil. Lipoic acid conjugate or dihydrolipoic acid conjugate (1.2 mmol) in acetonitrile or DMF was added into the above solution. A freshly prepared solution of trisodium citrate (3 to 10 mmol) in water (50 mL) was rapidly added into the above solution and the mixture refluxed for 30 minutes. The colloidal solution was cooled and kept for 2 to 3 days, and then filtered through a microporous filter (Millipore 0.22 μm filter) and centrifuged. The nanomaterial was resuspended in water and 2-propanol and centrifuged. The process of centrifugation and resuspension was carried out multiple times with water and 2-propanol to fully remove unreacted thiol.

Method 3: An aqueous golden yellow solution of HAuCl₄ 3H₂O (0.34 g, 1 mmol) and FeCl₃ (0.16 g, 1 mmol) in water (100 mL) was mixed with a solution of tetradecyl ammonium bromide (2.64 g, 4 mmol) in toluene (100 mL). The two-phase mixture was vigorously stirred for 60 minutes. Pentanethiol (0.13 g, 1.2 mmol) was then added, causing the organic phase to become orange. A freshly prepared sodium borohydride (0.38 g, 10 mmol) in water (25 mL) was slowly added (5 mL installment) with vigorous stirring which causes color changing to dark brown with evolution of gas. After complete addition, the reaction mixture was stirred for 3 hrs at 25 to 100° C. and the organic phase was isolated and evaporated to dryness. The resulting brown powder obtained was taken in toluene or chloroform (100 mL) and triturated with 2-propanol to precipitate the colloid and the product was collected by centrifugation and filtration. The residue was washed multiple times with 2-propanol to fully remove unreacted pentane thiol and stored at 4° C. The residue obtained was redistributed in acetonitrile or chloroform (100 mL) and into this solution dihydrolipoic acid conjugate (1 mmol) was added and the resulting mixture was stirred for 2 to 5 days. The reaction mixture was filtered and the residue obtained was washed thoroughly to remove unbound thiol.

1. A compound of the Formula A 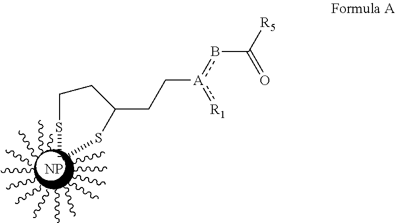
wherein: NP is gold nanoparticle; iron doped gold nanoparticle; Compound of the Formula A wherein S is bonded to one or more nanoparticle (NP) or direct bond between the two S atom bind to one or more nanoparticle (NP); A and B are carbon atoms directly connected to carbon-carbon single bond, carbon-carbon double bond; R₁ is (═O); —OH; —OR₂; —OR₃R₄; OR₄; —NH, —NR₂, —NR₃R₄, ═NH; ═NR₂, ═NR₃R₄; ═NR₄; —NHOH, —NOR₂, —NOR₃R₄, —NHOR₄, ═N—OH; ═NOR₂; ═NOR₃R₄; ═NOR₄; NHNHR₂; NHNHR₃R₄; NHNHR₄; NR₂SO₂R₂; NR₂SO₂R₃R₄; NR₂SO₂R₄; R₂ denotes COCH₃; —COCHCl₂; —COC₆H₅, -alkyl CₙH₂ₙ; -alkene CₘH₂ₘ; -alkyne CₘH₂ₘ₋₂; —(CH₂—CH₂O)ₙ CₘH₂ₘ₊₁; —(CH₂CH₂CH₂O)ₙ CₘH₂ₘ₊₁; —X(CH₂—CH₂O)ₙ CₘH₂ₘ₊₁Y; X(CH₂CH₂CH₂O)ₙ CₘH₂ₘ₊₁Y; n is 0-16; m is 0-16; X is CO, COO, CH₂O, CONHNH; Y is OH, NH₂, SH, COOH, SO₃H, H₂PO₄; R₃ denotes (CH₂)ₘ (XCH₂CH₂)ₙY; —(CH₂)ₘ (XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH; R₄ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol, Bevacizumab; R₅ is OH; OLi, ONa; OK, OMg, OMn, OCu, OCa, OAl, OFe, OAg, OAu, O-Niacinamide salt, O-Thiamine salt, ZR₆, ZR₇R₈, ZR₈, NR₉SO₂R₁₀ and Z is O, S, NH, NHNH; R₆ denotes -alkyl CₙH₂ₙ₊₂; -alkene CₘH₂ₘ; -alkyne CₘH₂ₘ₋₂; —(CH₂CH₂O)ₙCₘH₂ₘY; —(CH₂CH₂CH₂O)ₙ CₘH₂ₘY; n is 0-16; m is 0-16; Y is H, OH, NH₂, SH, COOH; R₇ denotes (CH₂)ₘ (XCH₂CH₂)ₙY; —(CH₂)ₘ (XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH; R₈ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol, Bevacizumab; R₉ denotes R₆, R₇R₈, R₈; R₁₀ denotes R₆, R₇R₈, R₈.

2. The compound of claim 1 wherein: NP is gold nanoparticle; iron doped gold nanoparticle; Compound of the Formula A wherein S is bonded to one or more nanoparticle (NP) or direct bond between the two S atom bind to one or more nanoparticle (NP); A and B are carbon atoms directly connected to carbon-carbon single bond, carbon-carbon double bond; R₁ is (═O); —OH; —OR₂; —OR₃R₄; OR₄; —NH, NR₂, —NR₃R₄, —NR₄, ═NH; ═NR₂, ═NR₃R₄; ═NR₄; —NHOH, —NOR₂, —NOR₃R₄, —NHOR₄, ═N—OH; ═NOR₂; ═NOR₃R₄; ═NOR₄; NHNHR₂; NHNHR₃R₄; NHNHR₄; NR₂SO₂R₂; NR₂SO₂R₃R₄; NR₂SO₂R₄; R₂ denotes COCH₃; —COCHCl₂; —COC₆H₅, -alkyl CₙH₂ₙ₊₁; -alkene CₘH₂ₘ; -alkyne CₘH₂ₘ₋₂; —(CH₂—CH₂O)ₙ CₘH₂ₘ₊₁; (CH₂CH₂CH₂O)ₙ CₘH₂ₘ₊₁; —X(CH₂—CH₂O)ₙ CₘH₂ₘ₊₁Y; X(CH₂CH₂CH₂O)ₙ CₘH₂ₘ₊₁Y; n is 0-16; m is 0-16; X is CO, COO, CH₂O, CONHNH; Y is OH, NH₂, SH, COOH, SO₃H, H₂PO₄; R₃ denotes (CH₂)ₘ (XCH₂CH₂)ₙY; —(CH₂)ₘ (XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH; R₄ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol, Bevacizumab; R₅ is OH; OLi, ONa; OK, OMg, OMn, OCu, OCa, OAl, OFe, OAg, OAu, O-Niacinamide salt, O-Thiamine salt, ZR₆, ZR₇R₈, ZR₈, NR₉SO₂R₁₀ and Z is O, S, NH, NHNH; R₆ denotes -alkyl CₙH₂ₙ₊₁; -alkene CₘH₂ₘ; -alkyne CₘH₂ₘ₋₂; (CH₂CH₂O)ₙ CₘH₂ₘY; —(CH₂CH₂CH₂O)ₙ CₘH₂ₘY; n is 0-16; m is 0-16; Y is H, OH, NH₂, SH, COOH; R₇ denotes (CH₂)ₘ (XCH₂CH₂)ₙY; —(CH₂)ₘ (XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH; R₈ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol, Bevacizumab; R₉ denotes R₆, R₇R₈, R₈; R₁₀ denotes R₆, R₇R₈, R₈.

3. A method of reducing the symptoms associated with free radical mediated diseases comprising administering an effective amount of the compound of the Formula A wherein: NP is gold nanoparticle; iron doped gold nanoparticle; Compound of the Formula A wherein S is bonded to one or more nanoparticle (NP) or direct bond between the two S atom bind to one or more nanoparticle (NP); A and B are carbon atoms directly connected to carbon-carbon single bond, carbon-carbon double bond; R₁ is (═O); —OH; —OR₂; —OR₃R₄; OR₄; —NH, —NR₂, —NR₃R₄, —NR₄, ═NH; ═NR₂, ═NR₃R₄; ═NR₄; —NHOH, —NOR₂, —NOR₃R₄, —NHOR₄, ═N—OH; ═NOR₂; ═NOR₃R₄; ═NOR₄; NHNHR₂; NHNHR₃R₄; NHNHR₄; NR₂SO₂R₂; NR₂SO₂R₃R₄; NR₂SO₂R₄; R₂ denotes COCH₃; —COCHCl₂; —COC₆H₅, -alkyl CₙH₂ₙ₊₁; -alkene CₘH₂ₘ; -alkyne CₘH₂ₘ₋₂; —(CH₂—CH₂O)ₙ CₘH₂ₘ₊₁; —(CH₂CH₂CH₂O)ₙ CₘH₂ₘ₊₁; —X(CH₂—CH₂O)ₙ CₘH₂ₘ₊₁Y; X(CH₂CH₂CH₂O)ₙ CₘH₂ₘ₊₁Y; n is 0-16; m is 0-16; X is CO, COO, CH₂O, CONHNH; Y is OH, NH₂, SH, COOH, SO₃H, H₂PO₄; R₃ denotes (CH₂)ₘ (XCH₂CH₂)ₙY; —(CH₂)ₘ (XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH; R₄ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol, Bevacizumab; R₅ is OH; OLi, ONa; OK, OMg, OMn, OCu, OCa, OAl, OFe, OAg, OAu, O-Niacinamide salt, O-Thiamine salt, ZR₆, ZR₇R₈, ZR₈, NR₉SO₂R₁₀ and Z is O, S, NH, NHNH; R₆ denotes -alkyl CₙH₂ₙ₊₁; -alkene CₘH₂ₘ; -alkyne CₘH₂ₘ₋₂; —(CH₂CH₂O)ₙ CₘH₂ₘY; —(CH₂CH₂CH₂O)ₙ CₘH₂ₘY; n is 0-16; m is 0-16; Y is H, OH, NH₂, SH, COOH; R₇ denotes (CH₂)ₘ(XCH₂CH₂)ₙY; —(CH₂)ₘ (XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH; R₈ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol, Bevacizumab; R₉ denotes R₆, R₇R₈, R₈; R₁₀ denotes R₆, R₇R₈, R₈.

4. A method of treating free radical mediated diseases comprising administering an effective amount of the compound of the Formula A and by hyperthermia therapy wherein: NP is gold nanoparticle; iron doped gold nanoparticle; Compound of the Formula A wherein S is bonded to one or more nanoparticle (NP) or direct bond between the two S atom bind to one or more nanoparticle (NP); A and B are carbon atoms directly connected to carbon-carbon single bond, carbon-carbon double bond; R₁ is (═O); —OH; —OR₂; —OR₃R₄; OR₄; —NH, —NR₂, —NR₃R₄, —NR₄, ═NH; ═NR₂, ═NR₃R₄; ═NR₄; —NHOH, —NOR₂, —NOR₃R₄, —NHOR₄, ═N—OH; ═NOR₂; ═NOR₃R₄; ═NOR₄; NHNHR₂; NHNHR₃R₄; NHNHR₄; NR₂SO₂R₂; NR₂SO₂R₃R₄; NR₂SO₂R₄; R₂ denotes COCH₃; —COCHCl₂; —COC₆H₅, -alkyl CₙH₂ₙ₊₁; -alkene CₘH₂ₘ; -alkyne CₘH₂ₘ₋₂; —(CH₂—CH₂O)ₙ CₘH₂ₘ₊₁; —(CH₂CH₂CH₂O)ₙ CₘH₂ₘ₊₁; —X(CH₂—CH₂O)ₙ CₘH₂ₘ₊₁Y; X(CH₂CH₂CH₂O)ₙ CₘH₂ₘ₊₁Y; n is 0-16; m is 0-16; X is CO, COO, CH₂O, CONHNH; Y is OH, NH₂, SH, COOH, SO₃H, H₂PO₄; R₃ denotes (CH₂)ₘ (XCH₂CH₂)ₙY; —(CH₂)ₘ (XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH; R₄ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol, Bevacizumab; R₅ is OH; OLi, ONa; OK, OMg, OMn, OCu, OCa, OAl, OFe, OAg, OAu, O-Niacinamide salt, O-Thiamine salt, ZR₆, ZR₇R₈, ZR₈, NR₉SO₂R₁₀ and Z is O, S, NH, NHNH; R₆ denotes -alkyl CₙH₂ₙ₊₁; -alkene CₘH₂ₘ; -alkyne CₘH₂ₘ₋₂; —(CH₂CH₂O)ₙ CₘH₂ₘY; —(CH₂CH₂CH₂O)ₙ CₘH₂ₘY; n is 0-16; m is 0-16; Y is H, OH, NH₂, SH, COOH; R₇ denotes (CH₂)ₘ(XCH₂CH₂)ₙY; —(CH₂)ₘ (XCH₂CH₂CH₂)ₙY; m=0-16, n=0-16, X is O, N, S, OCO, OCOO, NH, NHCO, NHCOO, OCH₂O, CONHNH; Y is O, N, S, OCO, OCOO, NH, NHCO, NHCOO, OCH₂O, CONHNH; R₈ denotes FITC, 7-amino-4-methyl-coumarin-3-acetic acid (AMCA), 4′,6′-Diamidino-2-phenylindole (DAPI), Lissamine, R-Phycocyanin, B-Phycoerythrin, Rhodamine, Tetramethylrhodamine isothiocyanate (TRITC), Texas Red, Biotin, Folic acid, Vitamin E, Pteroic acid, Leucovorin, Methotrexate, 5-FU, Taxol, PKKKRKV peptide, cRGDfK peptide, 6-Fluoroinositol; 6-Oxoinositol, Bevacizumab; R₉ denotes R₆, R₇R₈, R₈; R₁₀ denotes R₆, R₇R₈, R₈.

5. The method of claims 3 to 4 wherein the free radical mediated disease is cancer.

6. A pharmaceutical composition comprising a composition of any claims 1 through 5 and a pharmaceutically acceptable carrier.

7. The product of Formula A further comprising: a plurality of polyethylene glycol (PEG) molecules attached to the nanoparticle.

8. The product of Formula A further comprising: a plurality of alkane thiol molecules attached to the nanoparticle.

9. The product of the Formula A further comprising: a plurality of peptides containing cysteine attached to the particle.

10. The product of Formula A further comprising: a plurality of radioisotope molecules attached to the nanoparticle.

11. A method of imaging comprising: providing the multivalent product of claim 1; providing a subject to be imaged; contacting the multivalent product and the subject; and imaging said subject using the multivalent product.

12. The method of imaging claim 11, wherein said imaging comprises magnetic resonance imaging, fluorescence imaging, surfaced enhance Raman imaging, radiologic imaging, or the targeted delivery of radioisotopes.
